(19) United States
(12) Patent Application Publication
(10) Pub. No.: US 2015/0030544 A1
(43) Pub. Date: Jan. 29, 2015

(54) Enzymatic Nanosensor Compositions and Methods

(71) Applicant: Northeastern University, Boston, MA (US)

(72) Inventors: Heather A. Clark, Lexington, MA (US); Kevin Joseph Cash, Brighton, MA (US)

(21) Appl. No.: 14/383,036

(22) PCT Filed: Mar. 6, 2013
(86) PCT No.: PCT/US13/29396

§ 371 Date: Sep. 4, 2014

Related U.S. Application Data
(60) Provisional application No. 61/607,173, filed on Mar. 6, 2012.

(51) Int. Cl. C12Q 1/26; A61K 49/00; G01N 33/58; C12Q 1/32; C12Q 1/54

(52) U.S. Cl. CPC C12Q 1/26; C12Q 1/32; C12Q 1/54; G01N 33/582; A61K 49/005
USPC 424/9.6; 435/287.9; 435/25; 435/26; 435/20; 435/14

(57) ABSTRACT

Disclosed herein are compositions including a nanosensor that is sensitive to an analyte such that the nanosensor emits a fluorescent signal upon detecting the analyte, and a catalytic agent that catalyzes a reaction in which a target substrate is converted into one or more products, such that at least one of the one or more products is the analyte. In addition, methods of using the nanosensor-catalytic agent compositions to detect a target substrate are disclosed.

### CROSS REFERENCE TO RELATED APPLICATIONS

This application claims the benefit of U.S. Provisional Application No. 61/607,173, filed Mar. 6, 2012, the entire contents of which are hereby incorporated by reference herein.

### STATEMENT REGARDING FEDERALLY SPONSORED RESEARCH/DEVELOPMENT

This invention was made with government support by the Defense Advanced Research Projects Agency (DARPA) under award number W911NF-11-1-0025 and the National Institute of General Medicine of the National Institutes of Health under award number R01 GM084366. The government has certain rights in the invention.

### BACKGROUND

Target detection is an important component in biotechnology, analytical chemistry, analysis of environmental samples, and medical diagnostics. Certain types of detection assays, such as fluorescence-based assays, are capable of providing detailed pictures of where fluorescent molecules are localized in tissues and cells. In particular, fluorescence-based assays exhibit exceptional sensitivity, detecting small concentrations of fluorescent molecules.

In addition, direct, minimally invasive monitoring of in vivo physiological conditions presents a route to determine health status in real time and address needs as they arise. Current in vivo monitoring system designs are limited by invasive implantation procedures and bio-fouling, limiting the utility of these tools for obtaining physiologic data. Traditional approaches using enzymes as recognition elements primarily rely on the use of electrodes to read out the signal changes after target detection. This imposes a limitation for non-invasive or non-contact monitoring, as the electrode must be physically connected to instrumentation to be measured. Former approaches to nanosensors have been limited to targets, such as ions or small molecules, that can be extracted into the core of the nanosensors. This approach does not allow detection of larger targets and has limited capabilities of being extended to additional targets without significant costs to developing new extraction chemicals.

Thus, there is a need for compositions and methods for the inexpensive, sensitive, and rapid detection of a diverse range of biochemical targets.

### SUMMARY

Combining a catalytic agent with a fluorescent nanosensor that measures the effect of the enzymatic activity expands the range of detectable target substrates. The disclosed compositions and methods can be used in various contexts, including in biotechnology, analytical chemistry, analysis of environmental samples, and medical diagnostics. The disclosed methods and compositions can be used to detect targets in biological fluids, for cellular signaling, and for in vivo and in vitro monitoring. One application of the disclosed compositions and methods is to continuously track bioanalytes in vivo to enable clinicians and researchers to profile normal physiology and discover early markers for diseased states. Current in vivo monitoring system designs are limited by invasive implantation procedures and bio-fouling, which limit the utility of these systems for obtaining physiologic data. The disclosure allows measurement of a broad range of target substrates. Various combinations of fluorescent nanosensors and catalytic agents can be used to measure a wide range of target substrates both in vitro and in vivo.

According to aspects of the present disclosure, a composition includes a catalytic agent that catalyzes a reaction in which a target substrate and/or a co-substrate is converted into one or more products; and a nanosensor that is sensitive to an analyte such that the nanosensor emits a fluorescent signal upon detecting the analyte. The analyte is the target substrate, the co-substrate, or at least one of the one or more products.

In certain embodiments, the analyte includes oxygen, hydrogen, ammonia, nitrate, nitrite, and sulfate.

In further embodiments, the nanosensor is sensitive to oxygen. In other embodiments, the nanosensor includes a metal-centered dye, organic dye, or biological molecule. In other embodiments, the metal center of the metal-centered dye includes ruthenium (Ru(phen)3), platinum (Pt(II) meso-Tetra(pentafluorophenyl)porphine), osmium, rhenium, iridium, or mixtures thereof.

In some embodiments, the nanosensor and the catalytic agent are mixed together. In other embodiments, the nanosensor and catalytic agent are operably linked.

In some embodiments, the catalytic agent is diamino oxidase, acetylcholine esterase, glucose oxidase, cholesterol oxidase, or glutamate dehydrogenase.

In further embodiments, the nanosensor and catalytic agent are embedded in a matrix. In particular embodiments, the matrix is a hydrogel that allows the target substrate to contact the catalytic agent.

In further embodiments, the nanosensor and catalytic agent are attached to a surface of a microfluidic device. In other embodiments, the nanosensor and catalytic agent are attached to the surface of the microfluidic device through linkers.

In particular embodiments, the nanosensor and the catalytic agent are attached to the surface of a nanodevice. In some embodiments, the nanodevice includes a polymer to which the nanosensor and the catalytic agent are attached. In some embodiments, the polymer is polyvinyl chloride, polycaprolactone, polylactic acid, polylactic co-glycolic acid, poly(3-hydroxybutyrate), poly(carboxy phenoxy propane)-(sebacic acid), polypropylene fumarate, poly(alkyl cyanoacrylate, chitosan, alginate, polylysine, collagen, or mixtures thereof.

Aspects of the methods disclosed herein provide methods of detecting a target substrate, including contacting a catalytic agent with a target substrate and/or a co-substrate such that the catalytic agent catalyzes conversion of the target substrate and/or the co-substrate into one or more products. The methods also include contacting a nanosensor with an analyte such that the nanosensor emits a fluorescent signal upon detecting the analyte, wherein the analyte is the target substrate, the co-substrate, or at least one of the one or more products. The methods further include measuring the concentration of the target substrate based on the fluorescent signal generated by the nanosensor.

In certain embodiments, the methods include using analytes such as oxygen, hydrogen, ammonia, nitrate, nitrite, and sulfate. In certain embodiments, the methods include using a nanosensor that is sensitive to oxygen.

In certain embodiments, the nanosensors used in the methods include a metal-centered dye, organic dye, or biological molecule. In certain embodiments, the metal center of the metal-centered dye include ruthenium (Ru(phen)3), platinum (Pt(II) meso-Tetra(pentafluorophenyl)porphine), osmium, rhenium, iridium, or mixtures thereof.

In certain embodiments, the methods include using a nanosensor and a catalytic agent that are mixed together. In other embodiments, the nanosensor and the catalytic agent are operably linked.

In some embodiments, the catalytic agent used in the methods is diamino oxidase, acetylcholine esterase, glucose oxidase, cholesterol oxidase, or glutamate dehydrogenase. In other embodiments, the nanosensor and catalytic agent are embedded in a matrix. In some embodiments, the matrix is a hydrogel that allows the target substrate to contact the catalytic agent.

In further embodiments, the methods further include attaching the nanosensor and the catalytic agent to a surface of a microfluidic device. In further embodiments, linkers are used to attach the nanosensor and catalytic agent to the surface of the microfluidic device.

In further embodiments, the methods include attaching the nanosensor and the catalytic agent to a surface of a nanodevice. In certain embodiments, the nanodevice includes a polymer to which the nanosensor and the catalytic agent are attached. In further embodiments, the polymer is polyvinyl chloride, polycaprolactone, polylactic acid, polylactic co-glycolic acid, poly(3-hydroxybutyrate), poly(carboxy phenoxy propane)-(sebacic acid), polypropylene fumarate, poly(alkyl cyanoacrylate, chitosan, alginate, polylysine, collagen, or mixtures thereof.

### SHORT DESCRIPTION OF THE FIGURES

The following figures are presented for the purpose of illustration only, and are not intended to be limiting.

FIG. 1 shows a schematic of embodiments of the enzyme nanosensor compositions.

FIG. 2 shows the enzyme nanosensor response to histamine. While fluorescence from the nanosensors is low in the absence of histamine, addition of histamine consumes oxygen and increases sensor fluorescence.

FIG. 3 is a graphical representation of the enzyme nanosensor system responding rapidly and reversibly to histamine.

FIG. 4A is the same as FIG. 3 except that FIG. 4 includes all error bars, while FIG. 3 shows the errors bars from every five data points. FIG. 4B shows that cycling histamine levels without continuous excitation shows full reversibility. FIG. 4C shows that the nanosensors do not photobleach under continuous excitation in the in vivo animal imager. FIG. 4D represents the fluorescence spectrum from enzyme nanosensor reversibility.

FIG. 5 represents images from the in vitro calibration presented in FIG. 4.

FIGS. 6A-B are graphical representations showing that the enzyme nanosensor response is reproducible batch-to-batch. FIG. 6A shows that the absolute intensity of the sensors change slightly (about 10%), but FIG. 6B shows that the sensor response to histamine is not altered.

FIGS. 7A-B are graphical representations showing that altering the ratio of enzyme-to-nanosensor can control both the analyte response (FIG. 7A) as well as reaction kinetics (FIG. 7B).

FIG. 8 represents fluorescence data using glucose oxidase as the enzyme, enabling detection of the catalytic agent glucose.

FIGS. 9A-C represent in vivo experimental results that demonstrate the ability of intradermal enzyme nanosensor to continuously monitor fluctuating histamine levels.

FIGS. 10A-C represent fluorescence data for three animal experiments that demonstrate the ability of intradermal enzyme nanosensor to continuously monitor fluctuating histamine levels.

FIGS. 11A-B are graphical representations of all three histamine response curves (FIG. 11A) and averaged data (FIG. 11B, ±SD) for all three animal experiments.

FIG. 12 is a graphical representation showing that the enzyme nanosensor system responds rapidly to histamine concentrations in a dose-dependent manner.

FIG. 13 represents a one-compartment open model fit to the average in vivo data.

FIG. 14 represents microscopic images of the enzyme nanosensor composition (pH nanosensors and acetylcholinesterase) encapsulated in a microdialysis tube.

FIG. 15 is a graphical representation of fluorescence ratio of the nanosensors versus acetylcholine concentration, and shows that the sensors respond to acetylcholine in a dose-dependent manner.

FIG. 16 represents a calibration curve for oxygen nanosensors (with Pt(II) mess-Tetra (pentafluorophenyl)porphine as O₂ sensor dye and octadecyl rhodamine as the reference dye) combined with the catalytic agent glucose oxidase to detect glucose.

FIGS. 17A-B represent calibration curves similar to FIG. 16 except no reference dye was used and different catalytic agents were used. Glutamate oxidase was used for glutamate detection (FIG. 17A), and tyrosinase for dopamine detection (FIG. 17B).

FIG. 18 represents a calibration curve using oxygen-sensitive ultrasmall nanosensors with glutamate oxidase to detect glutamate.

### DETAILED DESCRIPTION

The patent and scientific literature referred to herein establishes knowledge that is available to those of skill in the art. The issued U.S. patents, allowed applications, published foreign applications, and references that are cited herein are hereby incorporated by reference to the same extent as if each was specifically and individually indicated to be incorporated by reference.

Although compositions and methods similar or equivalent to those described herein can be used in the practice or testing of the present invention, suitable compositions and methods are described below.

### DEFINITIONS

For convenience, certain terms employed in the specification, examples and claims are collected here. Unless defined otherwise, all technical and scientific terms used in this disclosure have the same meanings as commonly understood by one of ordinary skill in the art to which this disclosure belongs. The initial definition provided for a group or term provided in this disclosure applies to that group or term throughout the present disclosure individually or as part of another group, unless otherwise indicated.

In general, the compositions of the disclosure can be alternately formulated to comprise, consist essentially of, or consist of, any appropriate components disclosed in this disclosure. The compositions of the disclosure can additionally, or alternatively, be formulated so as to be devoid, or substantially free, of any components, materials, ingredients, adjuvants or species used in the prior art compositions or that are otherwise not necessary to the achievement of the function and/or objectives of the present disclosure.

The articles “a” and “an” are used in this disclosure to refer to one or more than one (i.e., to at least one) of the grammatical object of the article. By way of example, “an element” means one element or more than one element.

The term “or” is used in this disclosure to mean, and is used interchangeably with, the term “and/or,” unless indicated otherwise.

The present disclosure provides, in part, compositions that include a nanosensor that is sensitive to an analyte such that the nanosensor emits a fluorescent signal upon detecting the analyte; and a catalytic agent that catalyzes a reaction in which a target substrate is converted into one or more products, such that at least one of the one or more products is the analyte.

Aspects of the disclosed compositions comprise a catalytic agent and a fluorescent nanosensor. The fluorescent nanosensor measures the effect of the enzymatic activity, and expands the range of detectable target substrates. As disclosed herein, the compositions and methods are useful in biotechnology, analytical chemistry, analysis of environmental samples, and medical diagnostics. The disclosed compositions and methods can be used to detect targets in biological fluids, for cellular signaling, and for in vivo and in vitro monitoring. One application of the disclosed compositions and methods is to continuously track bioanalytes in vivo to enable clinicians and researchers to profile normal physiology and discover early markers for diseased states. In further embodiments, the disclosed compositions and methods detect analytes in environmental samples such as water samples (e.g., waste water, seawater, fresh water), soil samples, and samples from industrial production.

The disclosure allows measurement of a broad range of target substrates. Various combinations of fluorescent nanosensors and catalytic agents can be used to measure a wide range of target substrates both in vitro and in vivo.

Continuously monitoring in vivo substrate concentrations can be used in a wide range of applications, including but not limited to pharmacokinetic profiling of novel drugs or drug candidates and tracking biomarker concentrations during disease progression, treatment, or prevention. Current approaches rely on blood sampling followed by offline analysis. This process poses limitations when applied to common research models due to limitations on the amount and frequency of blood sampling.

In particular embodiments, the catalytic agent is an enzyme. Enzyme-based sensors can recognize a broad range of target substrates with high recognition specificity, but enzyme-based biosensors, including those for glucose, are still primarily based on electrochemical sensors. K. J. Cash, H. A. Clark, Trends Mol. Med 2010, 16. 584-593. In certain embodiments, the enzyme is an oxidase. For example, glucose oxidase catalytically oxidizes glucose into gluconic acid, which lowers the pH, and the measured pH change correlates to glucose concentration. However, any enzyme that catalyzes the reaction of one or more substrates to a product can be used.

Fluorescent nanosensors are a modular family of sensors that can continuously monitor in vivo physiological parameters, including but not limited to oxygen, pH, ammonia, nitrate, nitrite, and sulfate. The sensors are approximately 100 nm in diameter, and specific nanosensor formulations that emit a reversible, concentration-dependent fluorescent signal. In the present disclosure, incorporating catalytic agents with the nanosensors expands the range of detectable biological targets and constitutes a significant advance in the field of non-invasive continuous target substrate monitoring. In some embodiments, surface coatings (with, e.g., PEG domains) can minimize protein fouling and safely prolong nanoparticle clearance, and biocompatible polymers (e.g. PLGA) can also be used. Amongst other applications, this disclosure enables straightforward, minimally-invasive target substrate monitoring.

### Fluorescence Nanosensors

In the instant disclosure, a nanosensor is sensitive to an analyte such that the nanosensor emits a fluorescent signal upon detection of the analyte. In some embodiments, non-limiting examples of analytes include oxygen, hydrogen (pH), ammonia, nitrate, nitrite, and sulfate. Various fluorescent reports and derivatives thereof can be used in the disclosed compositions and methods. Nanosensors that are sensitive to oxygen include metal-centered dyes, organic dyes, and biological molecules. Metal-centered dyes include a combination of metals, ligand groups, or porphyrin. Non-limiting examples of metal-centered dyes include dyes with the following metals: ruthenium (for example, Ru(phen)3), platinum (for example, Pt(II) meso-Tetra(pentafluorophenyl)porphine)), osmium, rhenium, iridium, iridium, etc. Ligand groups that can be included in metal-centered dyes include phenanthroline; 2,2′-bipyridine; 4,4′-dicarboxy-2,2′-bipyridine; 4,7-diphenyl-1,10-phenanthroline; 2,2′-bipyridyl-4,4′-di-nonyl; 1,10-phenanthroline-5-amine; 1,10-phenanthroline-5-isothiocyanate; and 1,10-phenanthroline-5-N-hydroxysuccinimide ester. Porphyrin groups that can be included in metal-centered dyes include porphyrin, octaethylporphyrin ketone, tetra(pentafluorophenyl)porphine, octaethyl porphyrin, and coproporphyrin. Organic dyes include any dye quenched by O₂ and various fluorophores. Biological molecules include but are not limited to green fluorescent proteins (GFPs) and modified fluorescent proteins (FPs).

For the analyte hydrogen (for pH), fluorescent nanosensors can include fluorescein, chromoionophores, BCECF, 6-JOE, Oregon green (488, 514), pHrodo, SNARF (1, 4F, 5F), phenol red, biological (GFP and GFP mutants), and nanomaterials (QDs and carbon nanotubes, including with or without chemical modifications). Examples of fluoresceins include FITC/conjugated fluorescein, F12, F16, F18 (hydrocarbon tails), and PLGFA-fluorescein. Suitable chromoionophores include Chromoionophore I (i.e., 9-(Diethylamino)-5-(octadecanoylimino)-5H-benzo[a]phenoxazine), Chromoionophore II (i.e., 9-Dimethylamino-5-[4-(16-butyl-2,14-dioxo-3,15-dioxaeicosyl)phenylimino]benzo[a]phenoxazine), Chromoionophore III (i.e., 9-(Diethylamino)-5-[(2-octyldecyl)imino]benzo[a]phenoxazine), Chromoionophore VII (9-Dimethylamino-5-[4-(15-butyl-1,13-dioxo-2,14-dioxanonadecyl)phenylimino]benzo[a]phenoxazine), Chromoionophore IV (i.e. 5-Octadecanoyloxy-2-(4-nitrophenylazo)phenol), Chromoionophore X (i.e. 4-Dioctylamino-4′-(trifluoroacetyl)stilbene), Chromoionophore VI (4′,5′-Dibromofluorescein octadecyl ester), Chromoionophore VIII (3′,3″,5′,5″-Tetrabromophenolphthaleinethyl ester), Chromoionophore XVII (1-Hydroxy-4-[4-(2-hydroxyethylsulfonyl)phenylazo]naphthalene-2-sulfonic acid potassium salt), and Chromoionophore IX (4-Dibutylamino-4′-(trifluoroacetyl)stilbene).

For the analyte NH₃, fluorescent nanosensors can include the pH sensors disclosed herein, NH₃ reactive complexes, and nanosensors with ammonium ionophore.

For the catalytic agent NADH/NADPH, fluorescent nanosensors can include quantum dots, other semiconductor dyes such as carbon dots, thionine, methylene blue dyes, and other redox dyes.

Electroactive dyes include but are not limited to metal-centered dyes, methylene blue, ferrocene, thionine, and cytodhrome. Dyes for membrane potential include but are not limited to RH237, RH414, RH421, RH795, Di-4-ANEPPS, Di-8-ANEPPS, Di-2-ANEPEQ, Di-3-ANEPPDHQ, Di-12-ANEPPQ, and Di-4-ANEPPDHQ. Reference dyes can be used for any fluorophore that does not respond to the analyte of interest, or any fluorophore that has a different response. Other potential readout mechanisms include color change (absorbance), photoacoustics, MRI, CT, ultrasound, and reflectance.

In addition, detection of fluorescence can be accomplished using devices that can be obtained commercially from, for example, Molecular Devices, LLC, Sunnyvale, Calif.

### Encapsulation Methods

Encapsulation methods include but are not limited to using alginate beads, other hydrogel beads, polymer beads with double emulsion, and layer-by-layer assembled shells.

In some embodiments, the nanosensors and catalytic agent are mixed together without using linkage chemistry. For instance, the nanosensors and catalytic agents are mixed in a polymeric matrix such that the catalytic agent and nanosensors are embedded within the matrix. In certain embodiments, a plurality of nanosensors and catalytic agents are embedded in a polymeric matrix such as polylactic acid and polylactic (co-glycolic acid).

In other embodiments, the nanosensors and catalytic agent are operably linked. Linkage chemistries include using a wide range of available conjugation techniques, EDC/NHS, isothiocyanate, and click chemistry. Hermanson, Bioconjugate Techniques (2^{nd} edition) (2008). In certain embodiments, the catalytic agent is linked to the nanosensor. In certain embodiments, the nanoparticle is immobilized within a polymeric matrix that allows the substrate of interest through the matrix to the nanoparticle. For example, the nanoparticle can be embedded within a polylactic acid matrix. The polylactic acid matrix is then functionalized with a linker group such as a maleimide group. See, e.g., Yamashiro et al. (2008) Polymer Journal 40: 657-662. The maleimide group can then link the nanoparticle to the catalytic agent by, for instance, sulfhydryl crosslinking.

In addition, there are a variety of linker types that can be utilized to link catalytic agents and nanosensors. In some instances, photochemical/photolabile linkers, thermolabile linkers, and linkers that can be cleaved enzymatically can be used. Some linkers are bifunctional (i.e., the linker contains a functional group at each end that is reactive with groups located on the element to which the linker is to be attached). The functional groups at each end can be the same or different. Examples of suitable linkers that can be used include straight or branched-chain carbon linkers, heterocyclic linkers and peptide linkers. A variety of types of linkers are available from Pierce Chemical Company in Rockford, Ill. and are described in EPA 188,256; U.S. Pat. Nos. 4,671,958; 4,659,839; 4,414,148; 4,669,784; 4,680,338, 4,569,789 and 4,589,071, and by Eggenweiler, H. M, Pharmaceutical Agent Discovery Today 1998, 3, 552. NVOC (6 nitroveratryloxycarbonyl) linkers and other NVOC-related linkers are examples of suitable photochemical linkers (see, e.g., WO 90/15070 and WO 92/10092). Peptides that have protease cleavage sites are discussed, for example, in U.S. Pat. No. 5,382,513.

In some embodiments, the compositions include the nanosensor and catalytic agent embedded in a matrix. In certain embodiments, the matrix is a polymer selected from the group consisting of poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, silicones, polyalkylenes such as polyethylene, polypropylene, and polytetrafluoroethylene, polyalkylene glycols such as poly(ethylene glycol) (PEG), polyalkylene oxides (PEO), polyalkylene terephthalates such as poly(ethylene terephthalate), polyvinyl alcohols (PVA), and polyvinyl ethers. In certain embodiments, the polymer matrix has a shape. For example, the polymer matrix can be rectangular, spherical, tubular, oblong, elliptical, or irregular. Furthermore, the polymer matrix can be any size ranging from about 10 nm to about 100 mm.

In some embodiments, the matrix is a hydrogel that allows the target substrate to contact the catalytic agent. A “hydrogel” is a three-dimensional, semi-solid network of one or more polymers derived from monomers in which a relatively large amount of water is present in the wet state. A “gel” is a solvent-rich composition consisting of a solvent (imbibing solvent) in an insoluble, porous network comprising one or more polymeric organic molecules, where the solvent can be water, giving a “hydrogel,” a nonpolar organic solvent, giving “nonpolar gel” or a polar organic solvent or a solution of water and an organic solvent, giving a “semipolar gel.” One of ordinary skill in the art understands how to make and use hydrogels.

In certain aspects, the disclosed methods also include adding hydrogels comprising vinyl monomers, urea, formamide, polyethylene glycol, sugars, oligosaccharides, and polyvinylpyrolidone, and polyacrylamide. The gels can also include salts, buffers, or polypeptides to the pre-gelling solution, thereby regulating the viscosity, vinyl monomer diffusion during gel formation, interactions of the hydrogel polymer chains during gel formation, or degree of polymerization of the gelling solution.

In certain embodiments, the hydrogel can be given a particular shape. For instance, the hydrogel can be formed on a glass surface, and can be reacted with methacryloxypropyl trichlorosilane to bestow it with vinyl groups. In this case, a gel is formed in any particular shape, including but not limited to, rod, tube, sheet, cone, sphere, rectangle, square, or other shape allowed by a mold or environment. A gel can be formed as a sheet by pouring the gelling solution into a flat or curved mold, or between two plates.

According to aspects of the present disclosure, the nanosensors have a shape that allows for accurate measurement of an analyte, that is, emission of an accurate fluorescent signal upon detecting the analyte. In some embodiments, the nanosensors have a particular shape that provides a high surface-to-volume ratio that allows for accurate measurements. In some embodiments, the nanosensors has an oblong or rectangular shape. Exemplary shapes include rectangles, elongated cylinders having a diameter shorter than the length of the cylinder, oblong structures, parallelepiped structures, rhomboid structures, and elliptical structures. Generally, any structure that provides a high aspect ratio for the sensing agent is within the scope of the invention. By “high aspect ratio,” it is meant that the structures disclosed herein have lengths that are longer than their widths.

The disclosed nanosensors and catalytic agents can also be immobilized within multiwell plates. For example, the nanosensors can be conjugated to antibodies coating the surface of the multiwell plate. Tang et al. (2011) Biochemical Engineering Journal 53(2): 223-228. The nanosensors can also be attached to the surface of the wells of the multiwell plate using technologies described herein. The catalytic agents can also be attached to the surface of the wells of the multiwell plate using antibodies or linking technologies described herein.

### Catalytic Agents

Any catalytic agent that acts on a target substrate and changes the concentration of an analyte (for example, O₂, pH, electron transfer, etc.) can be used in the disclosed compositions and methods. Non-limiting examples of catalytic agents include diamino oxidase, acetylcholine esterase, glucose oxidase, cholesterol oxidase, monoamine oxidase, glutamate dehydrogenase, alcohol dehydrogenase, urease, creatininase, glutamate oxidase, glucose dehydrogenase, lactate oxidase, tyrosinase, 3α-hydroxysteroid dehydrogenase, and 11β-hydroxysteroid dehydrogenase.

### Microfluidic Devices

In other embodiments, the enzyme nanosensors are incorporated into a microfluidic device. Applications include using the device for sensing analytes in biological or non-biological fluids. In some embodiments, the nanosensor and catalytic agent are attached to a surface of a microfluidic device. In other embodiments, the nanosensor and catalytic agent are attached to the surface of the microfluidic device through linkers. In other embodiments, the nanosensor and the catalytic agent are attached to the surface of a nanodevice.

In other embodiments, the nanodevice includes a polymer to which the nanosensor and the catalytic agent are attached. Polymers useful in construction of the microfluidic device include but are not limited to polyvinyl chloride, polycaprolactone, polylactic acid, polylactic co-glycolic acid, poly(3-hydroxybutyrate), poly(carboxy phenoxy propane)-(sebacic acid), polypropylene fumarate, poly(alkyl cyanoacrylate, chitosan, alginate, polylysine, collagen, or mixtures thereof.

In certain embodiments, the polymer includes poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, silicones, polyalkylenes such as polyethylene, polypropylene, and polytetrafluoroethylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) (jointly referred to herein as “polyacrylic acids”), and copolymers and mixtures thereof, polydioxanone and its copolymers, polyhydroxyalkanoates, polypropylene fumarate), polyoxymethylene, poloxamers, poly(ortho)esters, poly(butyric acid), poly(valeric acid), poly(lactide-co-caprolactone), trimethylene carbonate, polyvinylpyrrolidone, and the polymers described in Shieh et al., 1994, J. Biomed. Mater. Res., 28, 1465-1475, and in U.S. Pat. No. 4,757,128, Hubbell et al., U.S. Pat. Nos. 5,654,381; 5,627,233; 5,628,863; 5,567,440; and 5,567,435. Other suitable polymers include polyorthoesters (e.g., as disclosed in Heller et al., 2000, Eur. J. Pharm. Biopharm., 50:121-128), polyphosphazenes (e.g., as disclosed in Vandorpe et al., 1997, Biomaterials, 18:1147-1152), and polyphosphoesters (e.g., as disclosed in Encyclopedia of Controlled Drug Delivery, pp. 45-60, Ed. E. Mathiowitz, John Wiley & Sons, Inc. New York, 1999), as well as blends and/or block copolymers of two or more such polymers. The carboxyl termini of lactide- and glycolide-containing polymers may optionally be capped, e.g., by esterification, and the hydroxyl termini may optionally be capped, e.g., by etherification or esterification. In certain embodiments, the polymer comprises or consists essentially of polyvinyl chloride (PVC), polymethyl methacrylate (PMMA) and decyl methacrylate or copolymers or any combination thereof.

In certain embodiments, the polymer includes a biocompatible polymer, e.g., selected from poly(caprolactone) (PCL), ethylene vinyl acetate polymer (EVA), poly(ethylene glycol) (PEG), poly(vinyl acetate) (PVA), poly(lactic acid) (PLA), poly(glycolic acid) (PGA), poly(lactic-co-glycolic acid) (PLGA), polyalkyl cyanoacrylate, polyethylenimine, dioleyltrimethyammoniumpropane/dioleyl-sn-glycerolphosphoethanolamine, polysebacic anhydrides, polyurethane, nylons, or copolymers thereof. In polymers including lactic acid monomers, the lactic acid may be D-, L-, or any mixture of D- and L-isomers. The terms “biocompatible polymer” and “biocompatibility” when used in relation to polymers are art-recognized. For example, biocompatible polymers include polymers that are neither themselves toxic to the host (e.g., a cell, an animal, or a human), nor degrade (if the polymer degrades) at a rate that produces monomeric or oligomeric subunits or other byproducts at toxic concentrations in the host.

The polymer may include a plasticizer, such as dioctyl sebacate (DOS), o-nitrophenyl-octylether, dimethyl phthalate, dioctylphenyl-phosphonate, dibutyl phthalate, hexamethylphosphoramide, dibutyl adipate, dioctyl phthalate, diundecyl phthalate, dioctyl adipate, dioctyl sebacate, Citroflex A4, Citroflex A6, Citroflex B6, Citroflex B4, or other suitable plasticizers. In certain embodiments, the plasticizer is poly(glycerol sebacate), PGS. In certain embodiments, e.g., particularly where the polymer is biocompatible, a biocompatible plasticizer is used. The term “biocompatible plasticizer” includes materials that are soluble or dispersible in the relevant polymer, which increase the flexibility of the polymer matrix, and that, in the amounts employed, are biocompatible. Suitable plasticizers are well-known in the art and include those disclosed in U.S. Pat. Nos. 2,784,127 and 4,444,933. Specific plasticizers include, by way of example, acetyl tri-n-butyl citrate (c. 20 weight percent or less), acetyltrihexyl citrate (c. 20 weight percent or less), butyl benzyl phthalate, dibutylphthalate, dioctylphthalate, n-butyryl tri-n-hexyl citrate, diethylene glycol dibenzoate (c. 20 weight percent or less) and the like.

Methods of fabricating microfluidic devices are known in the art. For instance, a microfluidic device can be made using soft lithography methods, microassembly, bulk micromachining methods, surface micro-machining methods, standard lithographic methods, wet etching, reactive ion etching, plasma etching, stereolithography and laser chemical three-dimensional writing methods, modular assembly methods, replica molding methods, injection molding methods, hot molding methods, laser ablation methods, combinations of methods, and other methods known in the art or developed in the future. A variety of exemplary fabrication methods are described in Fiorini and Chiu, 2005, “Disposable microfluidic devices: fabrication, function, and application” Biotechniques 38:429-46; Beebe et al., 2000, “Microfluidic tectonics: a comprehensive construction platform for microfluidic systems.” Proc. Natl. Acad. Sci. USA 97:13488-13493; Rossier et al., 2002, “Plasma etched polymer microelectrochemical systems” Lab Chip 2:145-150; Becker et al., 2002, “Polymer microfluidic devices” Talanta 56:267-287; Becker et al., 2000, “Polymer microfabrication methods for microfluidic analytical applications” Electrophoresis 21:12-26; U.S. Pat. No. 6,767,706 B2, e.g., Section 6.8 “Microfabrication of a Silicon Device”; Terry et al., 1979, A Gas Chromatography Air Analyzer Fabricated on a Silicon Wafer, IEEE Trans. on Electron Devices, v. ED-26, pp. 1880-1886; Berg et al., 1994, Micro Total Analysis Systems, New York, Kluwer; Webster et al., 1996, Monolithic Capillary Gel Electrophoresis Stage with On-Chip Detector in International Conference On Micro Electromechanical Systems, MEMS 96, pp. 491496; and Mastrangelo et al., 1989, Vacuum-Sealed Silicon Micromachined Incandescent Light Source, in Intl. Electron Devices Meeting, IDEM 89, pp. 503-506. Each of these references are incorporated herein by reference for all purposes.

In additional embodiments, the device is fabricated using elastomeric materials. Fabrication methods using elastomeric materials and methods for design of devices and their components have been described in detail in the scientific and patent literature. See, e.g., Unger et al., 2000, Science 288:113-16; U.S. Pat. No. 6,960,437 (Nucleic acid amplification utilizing microfluidic devices); U.S. Pat. No. 6,899,137 (Microfabricated elastomeric valve and pump systems); U.S. Pat. No. 6,767,706 (Integrated active flux microfluidic devices and methods); U.S. Pat. No. 6,752,922 (Microfluidic chromatography); U.S. Pat. No. 6,408,878 (Microfabricated elastomeric valve and pump systems); U.S. Pat. No. 6,645,432 (Microfluidic systems including three-dimensionally arrayed channel networks); U.S. Patent Application publication Nos. 2004/0115838, 2005/0072946; 2005/0000900; 2002/0127736; 2002/0109114; 2004/0115838; 2003/0138829; 2002/0164816; 2002/0127736; and 2002/0109114; PCT patent publications WO 2005/084191; WO 05030822A2; and WO 01/01025; Quake & Scherer, 2000, “From micro to nanofabrication with soft materials” Science 290: 1536-40; Xia et al., 1998, “Soft lithography” Angewandte Chemie-International Edition 37:551-575; Unger et al., 2000, “Monolithic microfabricated valves and pumps by multilayer soft lithography” Science 288:113-116; Thorsen et al., 2002, “Microfluidic large-scale integration” Science 298:580-584; Chou et al., 2000, “Microfabricated Rotary Pump” Biomedical Microdevices 3:323-330; Liu et al., 2003, “Solving the “world-to-chip” interface problem with a microfluidic matrix” Analytical Chemistry 75, 4718-23,” Hong et al, 2004, “A nanoliter-scale nucleic acid processor with parallel architecture” Nature Biotechnology 22:435-39; Fiorini and Chiu, 2005, “Disposable microfluidic devices: fabrication, function, and application” Biotechniques 38:429-46; Beebe et al., 2000, “Microfluidic tectonics: a comprehensive construction platform for microfluidic systems.” Proc. Natl. Acad. Sci. USA 97:13488-13493; Rolland et al., 2004, “Solvent-resistant photocurable “liquid Teflon” for microfluidic device fabrication” J. Amer. Chem. Soc. 126:2322-2323; Rossier et al., 2002, “Plasma etched polymer microelectrochemical systems” Lab Chip 2:145-150; Becker et al., 2002, “Polymer microfluidic devices” Talanta 56:267-287; Becker et al., 2000, and other references cited herein and found in the scientific and patent literature. Each of these references are incorporated herein by reference for all purposes.

In nanodevices, such as microelectromechanical systems (MEMS), the compositions can be incorporated in the nanodevice such that the device has surfaces coated with a catalytic agent that catalyzes the conversion of a target substrate and/or co-substrate into one or more products, and a nanosensor that is sensitive to an analyte and produces a fluorescent signal, where the analyte is a target substrate, a co-substrate, or at least one of the one or more products. In some embodiments, the nanosensors and catalytic agent are attached to the surface of a nanodevice. In other embodiments, the nanodevices includes a polymer to which the nanosensors and the catalytic agent are attached.

### Methods of Detecting a Target Substrate

The present disclosure relates to methods of detecting a target substrate. The methods include first contacting a catalytic agent with a target substrate and/or a co-substrate such that the catalytic agent catalyzes conversion of the target substrate and/or the co-substrate into one or more products. Next, the method includes contacting a nanosensor with an analyte such that the nanosensor emits a fluorescent signal upon detecting the analyte, wherein the analyte is the target substrate, the co-substrate, or at least one of the one or more products. The method then includes measuring the concentration of the target substrate based on the fluorescent signal generated by the nanosensors.

The methods use the various compositions disclosed in detail herein. The disclosed methods can be used various contexts, including in biotechnology, analytical chemistry, analysis of environmental samples, and medical diagnostics. The disclosed methods can be used to detect targets in biological fluids, for cellular signaling, and for in vivo and in vitro monitoring. One application of the disclosed methods is to continuously track bioanalytes in vivo to enable clinicians and researchers to profile normal physiology and discover early markers for diseased states. Current in vivo monitoring system designs are limited by invasive implantation procedures and bio-fouling, which limit the utility of these systems for obtaining physiologic data. The disclosure allows measurement of a broad range of target substrates. Various combinations of fluorescent nanosensors and catalytic agents can be used to measure a wide range of target substrates both in vitro and in vivo.

The following examples illustrate embodiments of the instant disclosure, but are not intended to limit the scope of the claimed invention. Alternative materials and methods may be utilized to obtain similar results.

### Examples

This Example describes compositions and methods used to increase the range of measurable analytes by combining a catalytic agent with a fluorescent nanosensor that measures the effects of the catalytic agent. The enzyme nanosensor compositions (for example, the enzyme diamino oxidase and oxygen nanosensors) are used to monitor in vivo the concentration of the histamine dynamics as the concentration rapidly increases and decreases due to administration and clearance. The enzyme nanosensor compositions measured kinetics that match those reported from ex vivo measurements. This Example establishes a modular approach to in vivo nanosensor design for measuring a broad range of potential target analytes. Replacing the catalytic agent, or both the catalytic agent and nanosensor, can produce a composition that measures a wide range of specific analytical targets in vitro and in vivo.

Histamine is an important biochemical intermediary in allergy and inflammation, neurotransmission, gastric disorders, chronic myelogenous leukemia, and bacterial signaling. Histamine measurements predominantly rely on discrete microdialysis or blood sampling followed by offline measurements such as HPLC. Although this approach functions adequately for some experiments, it does impose limitations on the ability to monitor histamine concentrations in real-time or in the absence of clinical laboratories for analysis, and suffers some of the same implantation drawbacks of electrode sensors. Mou et al., Biomaterials 2010, 31. 4530-4539. In vivo histamine concentrations vary over a wide range, from a resting plasma concentration as low as 4 nM (Bruce et al., Thorax 1976, 31. 724-729) to 240 μM in diseased states (Gustiananda et al., Biosensors & Bioelectronics 2012, 31. 419-425) and as high as hundreds of mM inside mast cells. (Graham et al., The Journal of experimental medicine 1955, 102. 307-18). Compositions and methods that can continuously monitor systemic histamine levels can help delineate event progression in basic biological processes such as allergic response and neurobiology as well as the improved developmental testing of drugs targeting the histamine pathway.

In this Example, the disclosure together the approach of enzyme recognition biosensors with optical nanosensors to enable continuous histamine tracking in vivo without the need for blood sampling. To validate the system, we measured and modeled histamine pharmacokinetics and compared them with established values from offline measurements. The nanosensor-based measurements matched established pharmacokinetic properties for in vivo histamine clearance without the time, expense, or difficulty of previously-used offline methods. More importantly, the histamine sensor shows that a modular enzyme-nanosensor design can continuously track small biomolecules in vivo. The use of alternate enzymes and nanosensors is contemplated in the instant disclosure, such that various sensors can be used for additional target substrates, including but not limited to acetylcholine and dopamine for in vivo and in vitro applications.

### Materials

Poly(vinyl chloride) (PVC), Bis(2-ethylhexyl) sebacate (DOS), tetrahydrofuran (THF), dichloromethane, Tris(4,7-diphenyl-1,10-phenanthroline)ruthenium(II) dichloride complex, and histamine dihydrochloride were purchased from Sigma Aldrich (St. Louis, Mo.). 5,10,15,20-Tetrakis(pentafluorophenyl)-21H,23H-porphine, platinum(II) (PtTPFPP) was purchased from Frontier Scientific (Logan, Utah). 1,2-disteroyl-sn-glycero-3-phosphoethanolamine-N-[methoxy(polyethylene glycol)-550] ammonium salt in chloroform (PEG-lipid) was purchased from Avanti Polar Lipids (Alabaster, Ala.). Diamine oxidase (DAO, 35 IU/mL) was purchased from Bio-Research Products Inc. (North Liberty, Iowa). Spectra/Por® In Vivo Microdialysis Hollow Fibers (13 kDa MWCO, 200 μm inner diameter) was purchased from Spectrum Laboratories, Inc. (Rancho Dominguez, Calif.). Epoxy (H2Hold) was purchased from ITW Performance Polymers (Riviera Beach, Fla.) and phosphate buffered saline (PBS, pH=7.4) was purchased from Life Technologies (Grand Island, N.Y.).

### Animal Research

All animal experiments were approved by the institutional animal care and usage committee (IACUC) of Northeastern University as well as the US Army Medical Research and Materiel Command (USAMRMC) Animal Care and Use Review Office (ACURO). The mice used in this research were male CD-1 Nude mice from Charles River (Wilmington Mass.). All experiments were carried out at Northeastern University.

### Nanosensor Fabrication

Oxygen nanosensors (O₂NS) were fabricated using methods previously reported for ion sensitive nanosensors. Dubach et al., Journal of visualized experiments: JoVE 2011; Dubach et al., Proc Natl Acad Sci USA 2009, 106. 16145-50. In brief, this process started with formulation of an optode dissolved in 500 μL THF comprising 30 mg PVC, 60 μL DOS, and 10.5 mg PtTPFPP. In a scintillation vial, 2 mg of PEG-lipid was dried and then re-suspended in 5 mL PBS with a probe tip sonicator for 30 seconds at 20% intensity (Branson, Danbury Conn.). 50 μL of the optode solution was diluted with 50 μL of dichloromethane, and the mixture was added to the PBS/PEG-lipid solution while under probe tip sonication (3 minutes, 20% intensity). The nanosensor solution was filtered with 0.22 μm syringe filter to remove excess polymer (Pall Corporation, Port Washington, N.Y.). Nanosensors were sized with a Brookhaven 90Plus (Holtsville, N.Y.) and had an effective diameter of approximately 100 nm. A rough estimate of particle concentration, based on Nanoparticle Tracking Analysis (NTA, Nanosight, Amesbury, UK) of a similar nanosensor preparation yields a concentration of ˜1.5×10¹² particles/mL. Enzyme nanosensor solution was prepared by mixing oxygen nanosensors with DAO solution (35 IU/mL) in a 1:1 volume ratio.

### In Vitro Characterization

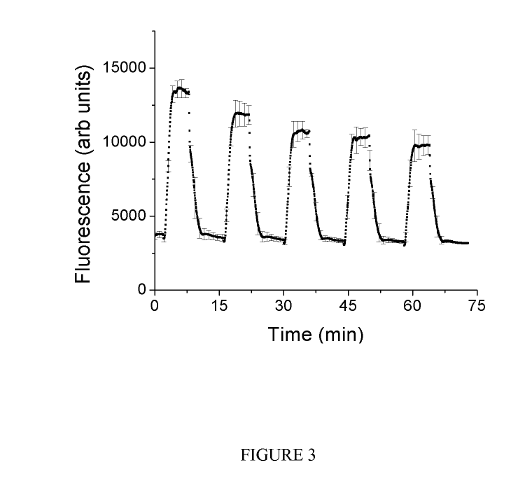

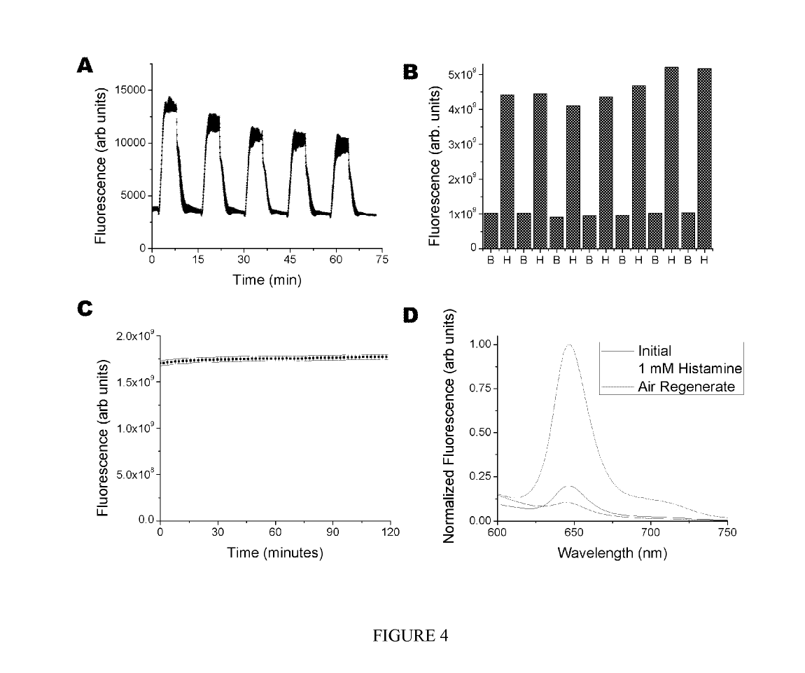

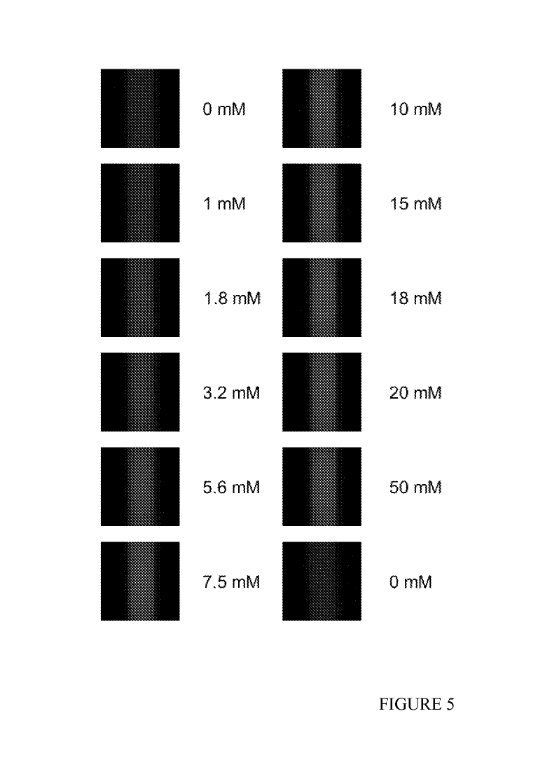

Enzyme nanosensor solution was loaded into microdialysis tubing via capillary action. The ends of the microdialysis tube were sealed with epoxy, and adhered to the bottom of a culture dish with an optical glass bottom. The setup was submerged in PBS for 1 hour to allow the epoxy to harden. All images were taken using a Zeiss confocal microscope (LSM 700) using 405 nm excitation and capturing emission above 612 nm using a 10× air objective. The histamine concentration was increased by addition of histamine stock solution (100 mM). Image analysis was performed using ImageJ. Intensity values were extracted from a three region of interest within the dialysis tubing which were averaged together. FIG. 5 are example images from the in vitro calibration presented in FIG. 4. Sensor affinity was determined with a dose response curve using OriginPro software (OriginLab, Northampton, Mass.) and the Hill1 fit. The limit of detection was determined as the concentration where the signal from the fit would be above 3 standard deviations from the blank signal. Reversibility cycling was conducted using a modified system with the microdialysis tubing affixed to a 20 mm glass coverslip loaded into a perfusion system on the microscope. Solutions of either 0 mM or 10 mM histamine were alternately filled into the system by gravity for a total of five cycles. This was repeated with three separate dialysis tubes in separate experiments. One region of interest was extracted from each experiment and these were averaged together. FIG. 3 shows the error bars for every five data points, while the full dataset is presented in FIG. 4.

FIG. 3 is a graphical representation of the enzyme nanosensors system responding rapidly and reversibly to histamine. After an addition of histamine (10 mM) to the nanosensors, the fluorescence rapidly increases (top of the response). Flushing the system with fresh buffer reverses the fluorescence change of the nanosensors, and is repeatable for several cycles of histamine detection (bottom of the response).

FIG. 4 represents further data and characterizations from the enzyme nanosensors system. FIG. 4A is the same as FIG. 3 except that FIG. 4 includes all error bars. FIG. 4B shows that cycling histamine levels without continuous excitation shows full reversibility. FIG. 4C shows that the nanosensors do not photobleach under continuous excitation in the in vivo animal imager. FIG. 4D represents the fluorescence spectrum from enzyme nanosensors reversibility.

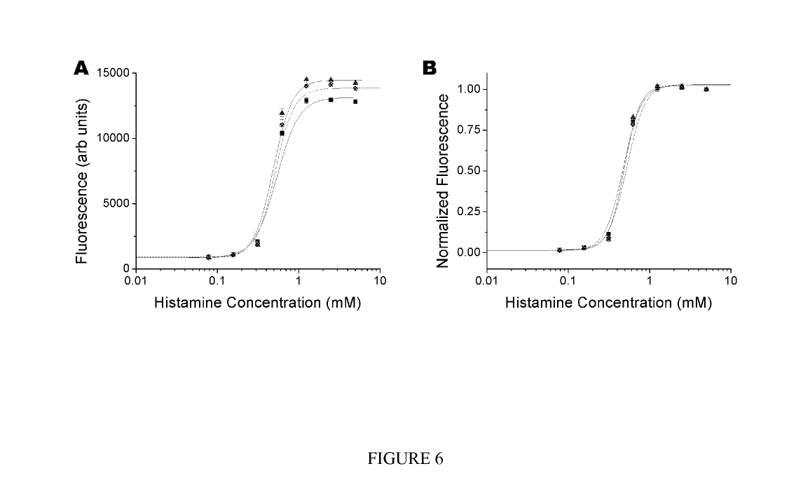

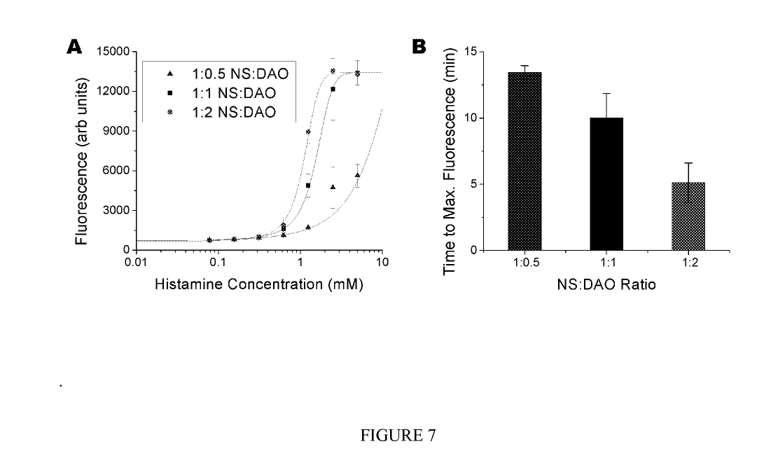

Additional in vitro characterizations, including photobleaching (FIG. 4), batch-to-batch variability (FIGS. 6A-B), enzyme ratio tuning (FIGS. 7A-B), as well as accompanying methods are described below.

### 1. Spectrum Characterization of Enzymatic Nanosensor Response

Fluorescence spectrum characterization of response and reversibility was performed with a QuantaMaster 40 from Photon Technology International (Birmingham, N.J.). 1.8 mL PBS was mixed with 400 μL of oxygen nanosensors and 1 mL of DAO solution (enzyme nanosensor) in a stirred quartz cuvette which was heated to 37° C. Fluorescence spectra were obtained exciting at 395 nm (5 nm slit) and collecting emission from 425-775 nm (5 nm slit) in 1 nm steps, at 0.1 sec integration/point and 3 scans per point averaged. 1 mM histamine was added and after the fluorescence peak signal had stabilized (˜60 minutes) another spectrum was obtained. Air was bubbled through the solution to reoxygenate the solution and determine sensor reversibility, and a final spectrum was obtained.

### 2. In Vitro Photobleaching

3 mL of oxygen nanosensors were placed in a sealed quartz cuvette and placed in the IVIS imager. They were exposed to continuous excitation and imaged every 2 minutes for 2 hours using the same imaging parameters as in vivo experiments.

### 3. Batch Reproducibility

To determine the inter-batch variability three separate optode solutions were fabricated and used to create three batches of oxygen nanosensors using the methods reported in the manuscript. The sizes of each batch by DLS were nearly identical (144 nm, PDI 0.19; 150 nm, PDI 0.18; 150 nm, PDI 0.18). Response characteristics of enzyme nanosensors made with these three batches were tested using a 96 well optical bottom plate. 200 μL of the enzyme nanosensor solution for each batch (DAO, oxygen nanosensors and PBS (volume 1:1:1)) was added to each well. The wells were scanned every minute using a Molecular Devices Gemini EM (Sunnyvale, Calif.) exciting at 395 nm, emission at 650 nm and a cutoff filter at 630 nm. After 30 minutes, 50 μL histamine stock solution was added to each well to raise the concentration to 0 nM, 20 nM, 200 nM, 2 μM, 20 μM, 200 μM, 2 mM and 20 mM (three wells at each concentration for each batch). The wells were then scanned every minute for 120 minutes using the same settings. Maximum intensity values were taken as the average response fluorescence (˜˜12 minutes after histamine addition) and used to generate the calibration curves. Data is also presented with the intensity normalized to the 20 mM data point for each batch. In both cases the data is fit with the Hill1 fit in OriginPro.

FIG. 6 shows that the enzyme nanosensors response is reproducible batch-to-batch. The absolute intensity of the sensors (FIG. 6A) change slightly (˜10%), but the sensor response to histamine is not altered (FIG. 6B). The dissociation constant (K_{d}) of the three batches were measured as 0.54 mM, 0.51 mM, and 0.49 mM; the slight difference between these values and those in FIG. 3 result from the different configuration of the microscope measurement system. The sizes of the oxygen nanosensors by DLS were 144 nm, 150 nm, and 150 nm.

### 4. Ratio Tuning

To study the impact on sensing of the ratio of enzyme to nanosensor on the calibration and response time, we prepared three enzyme nanosensor solutions at the following ratios 1:0.5:1.5, 1:1:1, 1:2:0 (NS:DAO:PBS). These solutions were then calibrated as with the batch reproducibility above with the additional data point of time to max fluorescence recorded and presented below.

FIGS. 7A-B show that altering the ratio of enzyme-to-nanosensor can control both the analyte response (FIG. 7A) as well as reaction kinetics (FIG. 7B). Decreasing the NS:enzyme ratio decreases the apparent K_{d} and the time to maximum fluorescence after histamine addition in an in vitro system.

5. Detection of Glucose with Enzymatic Nanosensors

As an example of the modular nature of the disclosed enzymatic nanosensors compositions and methods, we used glucose oxidase (GOx, Sigma) instead of the DAO to detect glucose instead of histamine. Oxygen nanosensors were combined with GOx (700 U/mL) in a 1:1 ratio and loaded into dialysis tubing and microscope perfusion setup as explained for histamine in the main methods section. Glucose solution (10 mM in PBS, pH 7.4) was perfused into the imaging chamber during imaging followed by a rest period, and then a PBS rinse to regenerate the initial signal.

### In Vivo Studies

All in vivo studies were conducted using a Lumina II in vivo imaging system (IVIS) from Caliper Life Sciences (Hopkinton, Mass.). A customized light source was used for excitation of the nanosensors built from 4 high intensity LEDs emitting at 395 nm (Newark Electronics, Chicago, Ill.) powered by a 9V battery. The IVIS was used in bioluminescence mode (no excitation light from the imager) with a 640 nm emission filter (20 nm bandpass) and 4 second exposure.

The O₂NS were concentrated approximately 10-fold for in vivo experiments using Amicon Ultra centrifugal filters (0.5 mL volume, 10 kDa MWCO, Millipore Corporation, Billerica, Mass.). Enzyme nanosensor solutions were prepared using concentrated O₂NS nanosensors (25 μL, ˜10¹³ particles) and DAO (50 μL, 1.75IU). As a control, O₂NS injections were made with concentrated nanosensors (25 μL) diluted with 50 μL of PBS. This control serves to measure changes in oxygen levels resulting from biological effects of histamine after injection (e.g. vasodilation, altered metabolism), and is necessary to enable specifically tracking histamine rather than a combination of histamine and oxygen changes. Mice were weighed, anesthetized with isoflurane (2% isoflurane, 98% oxygen), and placed in the IVIS imager. Two intradermal 30 μL injections of nanosensors were made along the midline of the back. Enzyme nanosensor was injected posterior to O₂NS. After injection the animals are imaged every 30 seconds for 30 minutes. After that, one mouse was administered 75 mg/kg histamine in PBS (i.p.) while the other mouse was administered PBS of a matching volume. The mice were imaged for an additional 45 minutes to 1 hour. All animals were sacrificed after the end of the experiment. Three separate experiments were performed with new mice and fresh batches of nanosensor solution. Sample images and timecourse data from all experiments are presented in the supplementary information.

For data analysis, a region of interest encompassing the injection area was selected and intensity was recorded. Each intensity value was normalized to the same spot at the first time point after injection of histamine. The difference in normalized signals between the enzymatic nanosensors and O₂NS was calculated for each mouse. This data was also averaged together across all three experiments using linear interpolation to align time and intensity points before averaging. Raw, normalized and averaged data is presented in the supplementary information. The average data was then fit to a single compartment open model: Equation (1)

$$I = A * \frac{{k}_{a}}{{k}_{a} - {k}_{e}} * [ {e}^{- {k}_{e} - ( t - {t}_{lag} )} - {e}^{- {k}_{a} - ( t - {t}_{lag} )} ] \qquad ( 1 )$$

Where I is the normalized fluorescent intensity difference, A is a scaling parameter, kₐ and kₑ are the absorption and elimination rate constants and t_{lag} is the lag time. The parameters kₐ, kₑ, and t_{lag} were fit using the method of residuals and A was fit using least squares minimization for plotting purposes.

### Results and Discussion

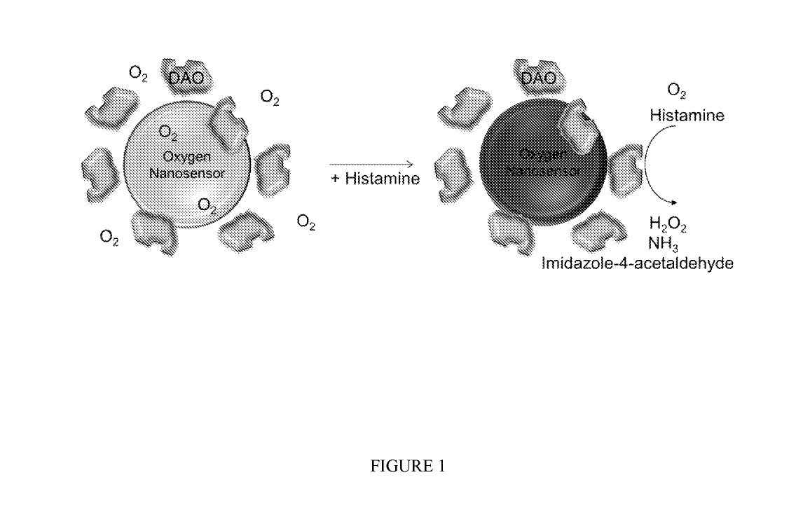

The modular platform for continuous optical biomolecule monitoring uses an enzymatic recognition element and fluorescent nanosensors. To translate the approach established with glucose oxidase-based electrochemical sensors, we selected an enzyme, diamino oxidase (DAO), that consumes oxygen when it coverts histamine into ammonia and imidazole-4-acetaldehyde. As shown in FIG. 1, when oxygen levels drop near active DAO, oxygen-responsive nanosensors (O₂NS) increase their fluorescence. In FIG. 1, the enzymatic recognition of histamine by diamine oxidase (DAO) reduces local oxygen concentration, increasing the fluorescence of oxygen sensitive nanosensors (O₂NS). A decrease in histamine concentration allows oxygen to return, decreasing fluorescence of the nanosensor. This approach of combining O₂NS with DAO detected histamine in both in vitro and in vivo experiments.

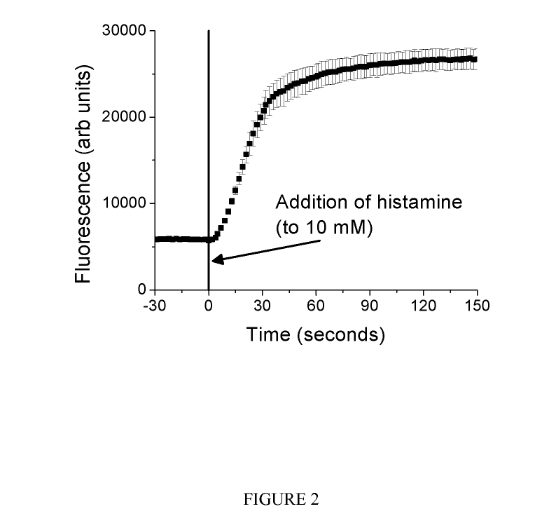

The O₂NS for this platform is a plasticized polymer nanoparticle core that contains Pt(II) meso-Tetra(pentafluorophenyl)porphine (PtTPFPP), a hydrophobic platinum porphyrin dye. Meier et al., Angewandte Chemie-International Edition 2011, 50. 10893-10896; Cywinski et al., Sensors and Actuators B-Chemical 2009, 135. 472-477; Borisov et al., Microchimica Acta 2009, 164. 7-15. These nanoparticles form through a well-established nanoemulsion technique, detailed in the methods section. Dubach et al., Journal of visualized experiments: JoVE 2011; Dubach et al., Nano Lett 2007, 7. 1827-31. PtTPFPP produces a reversible, oxygen-dependent fluorescent signal, and its ˜250 nm Stokes shift minimizes interference from tissue autofluorescence in vivo. When O₂NS come into contact with oxygen, the oxygen quenches nanosensor fluorescence, and the nanosensors recover their fluorescence once oxygen is removed from the environment. To make O₂NS sensitive to histamine, the sensor solution was mixed with a diamino oxidase (DAO) solution to form the enzyme nanosensor. In the absence of histamine, an air-saturated enzyme nanosensor solution emitted a low fluorescent signal, indicative of oxygen-induced quenching (FIG. 2). Upon addition of histamine, DAO consumes oxygen according to the following reaction:

Histamine+O₂+H₂O→imidazole-4-acetaldehyde+H₂O₂+NH₃

This reaction rapidly removes oxygen (t_{95%}=2.2 min, limited by mixing system) from the nanosensors, allowing the enzyme nanosensors to fluoresce. FIG. 2 shows the enzyme nanosensor response to histamine. Fluorescence from the nanosensors is low in the absence of histamine. Addition of histamine consumes local oxygen, increasing sensor fluorescence.

For longitudinal in vivo studies, enzyme nanosensor must change their fluorescence in a dose-dependent and reversible manner as histamine levels fluctuate. We demonstrated that enzyme nanosensors are reversible by encapsulating enzyme nanosensors in microdialysis tubing, washing through several cycles of histamine solutions or histamine-free buffer, and measuring the fluorescence with a confocal microscope. The enzyme nanosensor cannot diffuse across the tube walls, but small molecules such as histamine and oxygen can easily diffuse across the tube wall. Through 5 wash cycles and nearly 75 minutes of imaging, enzyme nanosensor reversed and settled to steady-state fluorescent intensities at each cycle (FIG. 3). Although the continuous laser excitation on the confocal microscope induced some photobleaching, the weaker light source used for in vivo experimentation did not cause a discernible loss of fluorescence (FIG. 4). In vivo, the vasculature will continuously supply oxygen to the nanosensors, ensuring that in the absence of oxygen-consuming enzymatic activity, enzyme nanosensor will reliably return to a quenched state. Furthermore, the enzyme nanosensor dose-response behavior in response to histamine solutions ranging from 1 to 50 mM, fit the Hill binding model well (FIG. 4) with a K_{d} of 3.4 mM and a lower limit of detection of 1.1 mM.

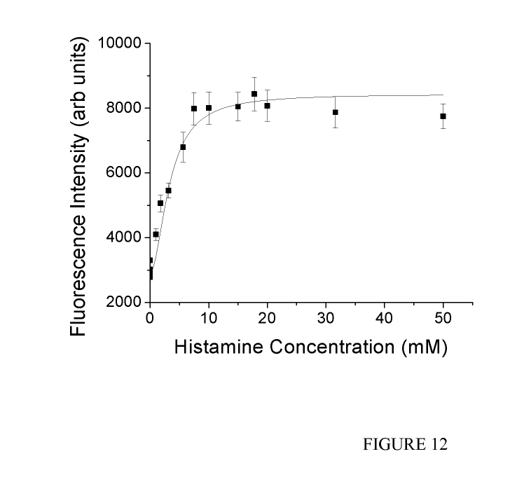

In FIG. 12, the graphical data show that the enzyme nanosensor system responds rapidly to histamine concentrations in a dose-dependent manner. As histamine concentration is increased, the fluorescence from the nanosensors increases with an apparent binding constant of 3 mM.

In vivo testing is a common failure point for sensing platforms because proteins may adsorb and foul the sensor, similar biomolecules may produce false positive signals, and normal oxygen fluctuations may mask the sensor's response. For in vivo tests, a whole animal imaging system continuously measured the enzyme nanosensor fluorescence in response to changes in systemic histamine. Anesthetized mice received two injections along the centerline of their back; one site for enzyme nanosensor and one site for enzyme-free O₂NS. The O₂NS measured systemic oxygen and thus can account for any changes in blood oxygenation or skin optical density as a result of histamine-induced vasodilation. Church et al., Journal of Allergy and Clinical Immunology 1997, 99. 155-160. By analyzing fluorescent dynamics from both spots, an accurate histamine measurement is possible even with concurrent changes in oxygen concentration.

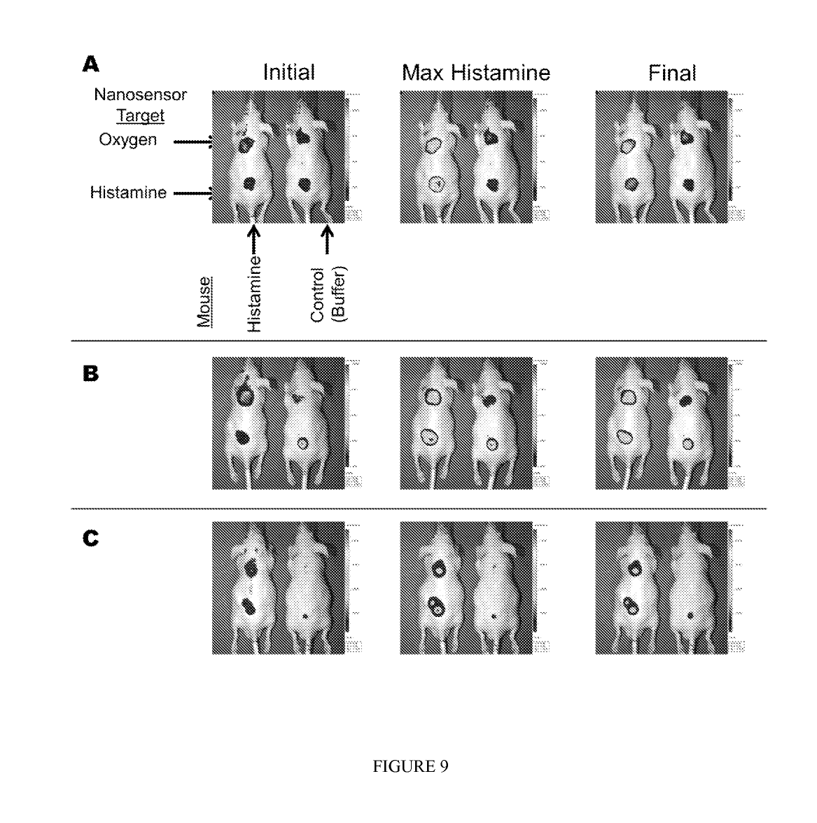

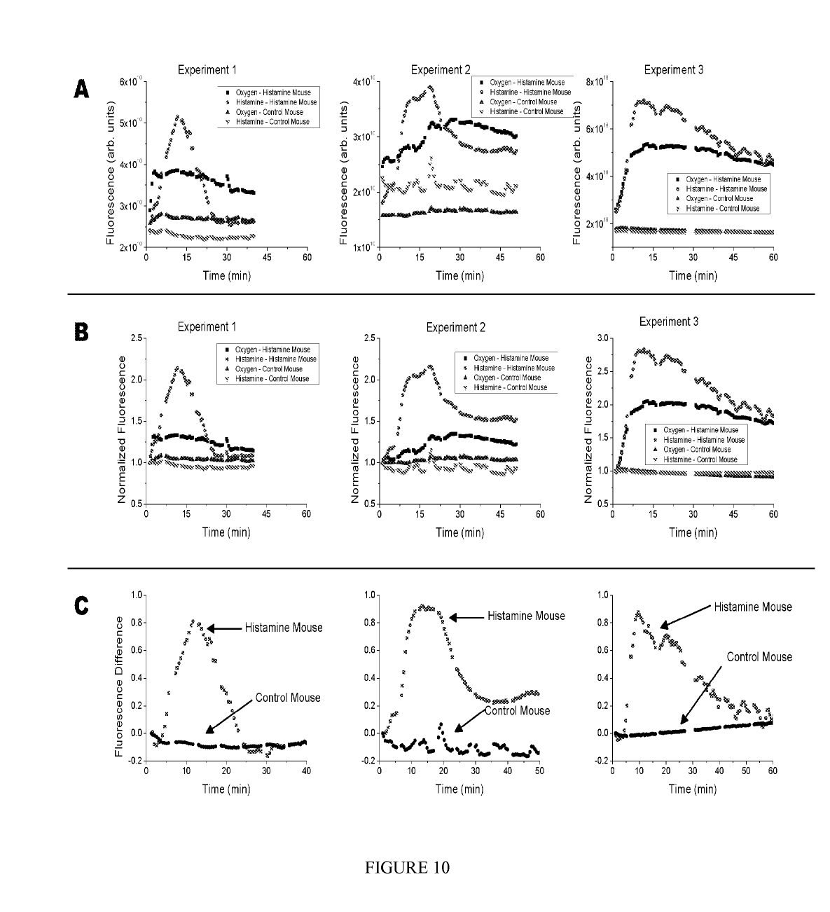

When the mice received an intraperitoneal histamine injection, the enzyme nanosensor implantation site fluoresced more brightly by a factor of 2.1 as it responded to histamine (FIG. 9A, left mouse, lower spot). The O₂NS implantation site (upper spot) also increased its fluorescence, although the increase was only ˜25% as large as the increase from the enzyme nanosensor spot. For control mice, who received saline rather than histamine, neither the enzyme nanosensor nor the O₂NS injection spots changed throughout the course of the experiment. FIG. 10C (Experiment 1) shows a normalized intensity plot that corrects for the effects of increased oxygen, measured by the O₂NS, showing a clear difference between the control mouse and the histamine mouse that peaks after 12 minutes. After approximately 30 minutes, the enzyme nanosensor returned to basal fluorescence and the two signals from control (saline) and test (histamine) mice were equal (FIG. 10C).

FIG. 9 represent in vivo experimental results that demonstrate the ability of intradermal enzyme nanosensor to continuously monitor fluctuating histamine levels. The figures demonstrate the return to baseline fluorescence after histamine clearance (rightmost image). FIGS. 9A-C represent images from three animal experiments demonstrating a similar trend for histamine dynamics. Sensor injections and mouse position are the same in each of the three experiments. As histamine levels increase (via i.p. injection), enzyme nanosensor fluorescence drastically increases (left mouse, bottom injection), while the O₂NS (top injection, controlling for oxygenation effects) shows a much smaller increase. As histamine levels decrease, the enzyme nanosensor fluorescence decreases as well. No signal change is seen from the control mouse (right mouse). The differential fluorescence between the two sensor sites (enzyme nanosensor and O₂NS) demonstrates the response of the nanosensors to histamine levels (far right).

FIG. 10 represents fluorescence data for all three animal experiments. FIG. 10A represents raw intensity values for each of the nanosensor injections (EnzNS and O2NS for both histamine and control mouse) in the three experiments. FIG. 10B represents fluorescent intensity values for each of the nanosensor injections normalized to the first data point after histamine injection for the three experiments. FIG. 10C represent differential fluorescence intensity values for the three experiments.

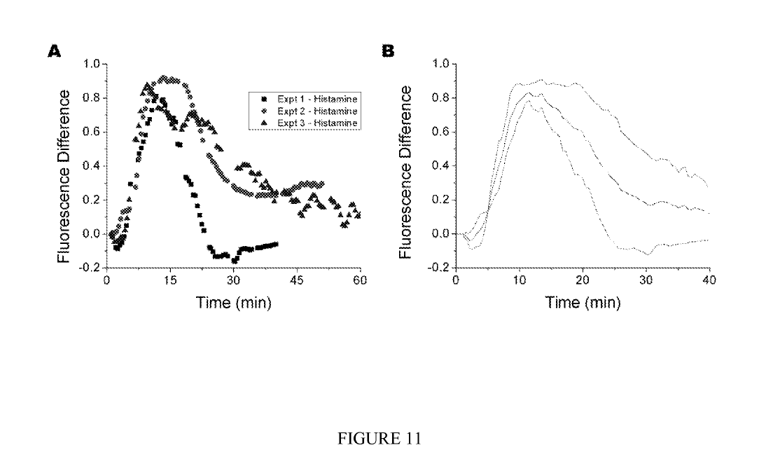

FIGS. 11A-B are graphical representations of all three histamine response curves (FIG. 11A) and averaged data (FIG. 11B, ±SD) for all three animal experiments.

This kinetic profile agrees with off-line measurement studies that have documented rapid rates for histamine clearance. Petersen et al., Journal of Allergy and Clinical Immunology 1996, 97. 672-679; Pollock et al., Agents and Actions 1991, 32. 359-365; Sakurai et al., Journal of Pharmacological and Toxicological Methods 1993, 29. 105-109. Running this experiment in triplicate demonstrated the reproducibility for detecting histamine using this approach. All three experiments showed similar response kinetics (see supporting information FIGS. 9-11), with biological variation likely accounting for differences. Averaged data from the three experiments fit into a single compartment open model for pharmacokinetics (Equation (1), described in the methods) indicating an approximate absorption half-life of 2.8 minutes and an elimination half-life of 7.6 minutes (FIG. 6). Other studies that measured histamine in humans using offline techniques yield elimination half-lives ranging from 4 minutes to 18 minutes. Petersen et al., Journal of Allergy and Clinical Immunology 1996, 97. 672-679; Pollock et al., Agents and Actions 1991, 32. 359-365; Middleton et al., J. Clin. Pharmacol. 2002, 42. 774-781. These data indicates that the enzyme nanosensor system accurately tracked histamine levels as it was cleared from the mice.

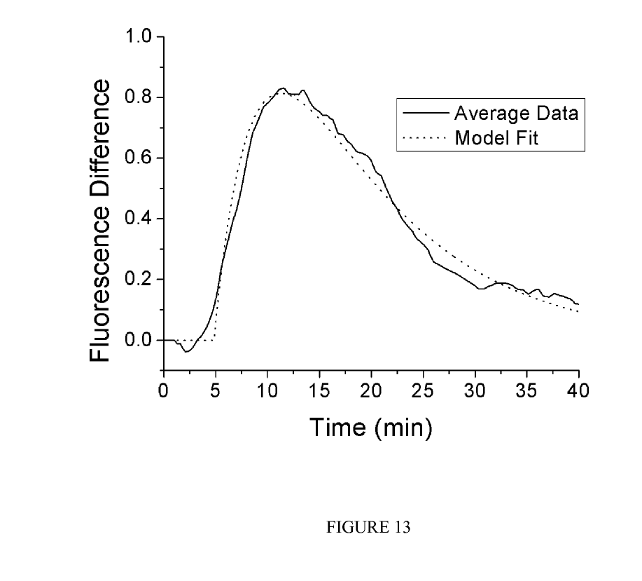

FIG. 13 represents a one-compartment open model fit to the average in vivo data. The model parameters yield an elimination half-life of 7.6 minutes, an absorption half-life of 2.8 minutes and a lag time of 4.8 minutes. This data matches well with available literature values. Petersen et al., Journal of Allergy and Clinical Immunology 1996, 97. 672-679; Pollock et al., Agents and Actions 1991, 32. 359-365; Sakurai et al., Journal of Pharmacological and Toxicological Methods 1993, 29. 105-109; Middleton et al., Clin. Pharmacol. 2002, 42. 774-781.

Traditional in vivo bio-analytical measurement systems have relied on electrochemical detection due to the robust and modular nature of enzyme recognition elements and the sensitivity of electrochemical measurement systems. These systems are useful for ex vivo measurements, but several factors will continue to confound their effectiveness in vivo. Primarily, electrode implantation produces local inflammation and induces a foreign body response with the eventual fate of fibrous capsule formation. Frost et al., Analytical Chemistry 2006, 78. 7370-7377. The fibrous capsule limits mass transfer near the electrode, changing measurement profiles, and every new electrode implantation introduces a new potential infection site. Although advances in wireless communications (Chang, et al., The Analyst 2012, 137. 2158-65; Vaddiraju, et al., Biosensors & Bioelectronics 2010, 25. 1553-1565) and supporting electronics may reduce the risk for infection, the foreign body response will still lead to capsule formation and performance loss in signal fidelity.

Nanoparticles implanted by subcutaneous injections minimize the complications from infection risk and capsule formation, and the Enzyme nanosensor nanoparticles are coated with poly(ethylene glycol) (PEG) to minimize protein fouling. Owens et al., Int. J. Pharm. 2006, 307. 93-102. This coating allows the nanosensors to provide a continuous signal with minimal side effects. Continuous, non-invasive physiological monitoring is extremely beneficial for longitudinal analyte monitoring in patients with chronic conditions such as diabetes or renal failure as well as in laboratory research. This monitoring is especially valuable for experiments using transgenic mouse models where the number of potential blood samples is limited and the cost per animal is very high, which precludes high temporal resolution for tracking analyte concentrations. In a clinical application, a patient would receive a tattoo-like subdermal injection with a spatially-multiplexed pattern so that each spot would monitor one of several analytes important to maintaining a positive prognosis.

One of the biggest advantages of embodiments of the invention is the modular nature of the combination of nanosensor and enzyme. Previous optode-based nanosensor formulations relied on the range of available ionophores or boronic acids as recognition element limits. Until now, those nanosensors were limited in the breadth of potential analytes by the available recognition elements. In the instant embodiments, those same nanosensors detected an enzyme's activity, making the resulting optical signal specifically responsive to the enzyme's target substrate. Embodiments of the invention increase the breadth of target analytes, which can include many more molecules due to the specific recognition capabilities intrinsic to enzymes

### Summary

Long-term physiologic monitoring requires continuously tracking in situ histamine levels, or those of any analyte, and this requires that the sensor fluorescence and response change only negligibly over the course of tracking Nanosensors and enzymes are both sufficiently small to diffuse away from the injection site. The nanosensors will not only track histamine levels in vivo for long enough to observe a return to basal levels, but also will require the sensor system to stay at the injection site for extended lengths of time. Rather than using spherical sensors as with this work, high aspect ratio sensors show significantly slower diffusion rates and keep sensors near the injection site longer. Ozaydin-Ince et al., Proc. Natl. Acad. Sci. U.S.A. 2011, 108. 2656-2661.

The sensor lifetime and long-term biocompatibility are important for prolonged analyte monitoring. Directly conjugating the enzyme to the sensor surface or co-encapsulating the enzyme and nanosensor will keep the platform intact and functional for a longer period of time. This linkage may also increase the sensitivity of the sensor system through more localized oxygen depletion which will in turn lower the minimal detection limit. Many important biomolecules have substantially lower physiological concentrations than the mM levels in this study, and working in the nanomolar or low micromolar range would make detection of targets such as cortisol and other hormones feasible. Another important step towards longitudinal monitoring is the incorporation of a reference fluorophore that is not sensitive to oxygen concentrations, which will enable ratiometric measurements. The fluorescence ratio of the two fluorophores will change with oxygen, or in this case histamine, concentrations, but will not depend on sensor concentration as the current approach does. The current approach tracks changes in histamine levels, but the use of ratiometric measurement opens up the possibility of absolute quantification of histamine concentrations in vivo. The ratio of enzyme:sensor also contributes to the platform's sensitivity, and varying that ratio is an auxiliary factor to realize a highly sensitive bio-analytical sensor.

In summary, we produced optical, enzyme-based nanosensor systems to monitor target substrates, such as histamine, in vivo. The enzyme nanosensor platform combined enzymatic biorecognition by diamino oxidase with oxygen sensitive nanosensors that produce a fluorescent signal visible through the mouse's skin. A dose-response calibration curve and time-course imaging experiments showed that enzyme nanosensor are reversible and sensitive in a physiologically-relevant concentration range. We then were able to continuously monitor systemic histamine concentrations in live mice, observing an increase from the histamine dose and then return to normal levels as histamine cleared the mice. Measurements based on enzyme nanosensor fluorescence matched the known elimination kinetics for histamine, indicating that this system accurately tracks histamine dynamics in vivo. Future work will produce new sensors based on this modular platform by replacing the recognition enzyme or replacing both the enzyme and nanosensor as well as directly conjugating the enzymes and nanosensors together. These sensors will enable simultaneous and continuous physiologic measurements for a wide range of analytical targets, and those measurements can establish standards for basal and perturbed health conditions which are difficult to attain with current monitoring techniques.

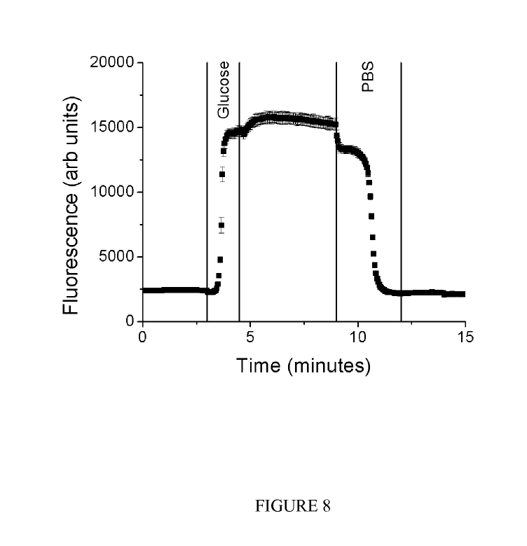

This Example discloses histamine as an important biomolecule to allergies and anaphylaxis. However, it is contemplated that this modular platform can quantifiably monitor other biologically important small molecules such as but not limited to lactate, creatinine and urea. Any of these designs are achievable by replacing diamino oxidase enzyme with an oxidase enzyme for the desired target. FIG. 8 demonstrates this embodiment with an alternate enzyme, for example, glucose oxidase. In FIG. 8, using glucose oxidase instead of diamino oxidase enables the detection of glucose instead of histamine. FIG. 8 shows the detection of 10 mM glucose, as well as a reversible fluorescent signal.

If an oxidase enzyme is unavailable or ineffective for a desired target, the platform can support a pair of two complimentary enzymes along with the oxygen nanosensors. In such a case, a suitable primary enzyme to the target analyte would be coupled with a secondary oxidase enzyme that targets a breakdown product or co-substrate from the primary reaction. Nanosensors can be fabricated for a wide range of products to measure based on commercially-available ionophores including ammonium, nitrate, carbonate or pH. This is the first work to demonstrate in vivo the principle of enzyme coupled optical nanosensors for histamine detection, and to tune the nanosensors to match their dynamic range to physiological levels for in vivo detection.

Different catalytic agent/nanosensors combinations can be used to detect various target substrates. The compositions to detect histamine and glucose have been discussed herein. Other tested combinations are listed in Table 1 below.

TABLE 1
Catalytic Agent/Nanosensor Combinations to Detect Target Substrates
Target Substrate/Catalytic Agent/Nanosensor Analyte/Fluorophore

Histamine/DAO/O₂/Ru
DAO: diamino oxidase
Ru: Tris(4,7,diphenyl-1,10-phenanthroline)Ru(II)Cl2
Histamine/DAO/O₂/Pt
Pt: Pt(II) meso-Tetra(pentafluorophenyl)porphine
Glucose/GOx/O₂/Pt
GOx: glucose oxidase
Glucose/GOx/O₂/Pt (UNS)
UNS: ultrasmall nanosensors
UNS are nanosensors fabricated in a different method using
surfactant micelles to template silica instead of plasticized
polymer.
Acetylcholine/Acetylcholinesterase/pH/DAF & Rh18
DAF: diamino fluorescein
Rh18: octadecyl rhodamine
ACh/AChE/pH/PLGA-Fl, PLGA-Rh
PLGA with fluorescein or rhodamine attached
Cholesterol/COx/O₂/Ru
COx: cholesterol oxidase
Alcohol (&acetylaldehyde)/YADH/NADH
YADH: yeast alcohol dehydrogenase
Alcohol (& acetyaldehyde)/YADH/NADH/Thionine
Alcohol (& acetyaldehyde)/YADH/NADH/MB
MB: methylene blue
Alcohol (& acetyaldehyde)/YADH/NADH/Peredox/mcherry
Peredox/mcherry is a protein from another lab which
senses NADH concentrations.
Lactate/Lactate oxidase/O₂/Ru
Ru: Tris(4,7,diphenyl-1,10-phenanthroline)Ru(II)Cl2
Androsterone/3AHSD/NADH/QDs
3alpha hydroxysteroid dehydrogenase
QD: quantum dots
Urea/Urease/pH/PLGA-Fl, PLGA-Rh
Urea/Urease/pH/CHIII/Rh18
CHIII: chromoionophore III
Creatinine/multiple/pH/PLGA-FL, PLGA-Rh
Multiple: creatininase, creatinase, urease
Creatinine/multiple/pH/CHIII/Rh18
Multiple: creatininase, creatinase, urease
Glutamate/glutamate oxidase/O2/Pt (reg & UNS)
Dopamine/tyrosinase/O₂/Pt
Glucose/GOx/O₂/Pt & Rh18
Encapsulation
Alginate beads
Androsterone/3AHSD/NADH
Double emulsion
Only Dextran-FITC (not enzyme or sensor)
Linkage Chemistry
Conjugate Ru to enzymes via NHS chemistry
Cholesterol oxidase - Ruthenium
Glucose oxidase - Ruthenium

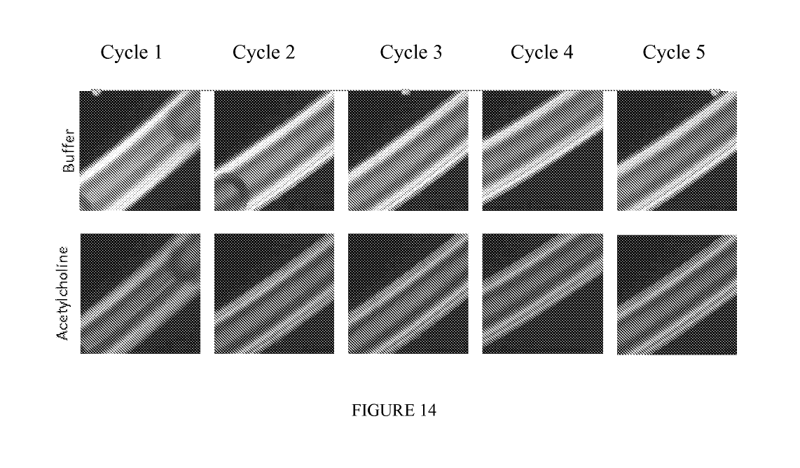

FIGS. 14-18 represent data for additional catalytic agent/nanosensors combinations used to detect target substrates. FIG. 14 represent microscopic images of the enzyme nanosensors composition (pH nanosensors and acetylcholinesterase) encapsulated in a microdialysis tube to detect acetylcholine. From left to right, different cycles of buffer (top) and acetylcholine solution (bottom) are shown. FIG. 14 shows that the response is reversible after acetylcholine exposure and can repeat for at least 5 cycles. DAF and Rh18 were the fluorophores used. Detection of acetylcholine was based on a similar methods to that of histamine and idaminooxidase. In the presence of acetylcholine, the enzyme degraded to choline and acetic acid, lowering the pH. The nanosensors and enzyme were encapsulated in a microdialysis fiber and imaged using confocal microscopy. Addition of acetylcholine lowered the pH, changing the fluorescence. Replacing the solution with fresh buffer generated the initial signal. The process was repeated for five cycles, showing that the process was reversible for at least several cycles.

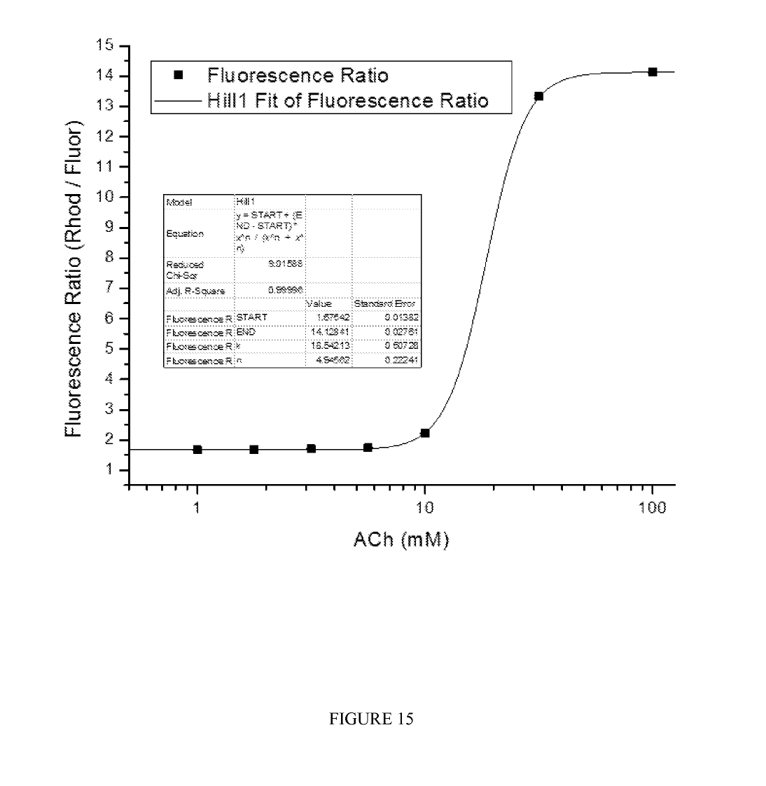

FIG. 15 is a graphical representation of a calibration of fluorescence ratio of the nanosensors versus acetylcholine concentration, and shows that the sensors respond to acetylcholine in a dose-dependent manner. PLGA-FI and PLGA-Rh were the fluorophores used.

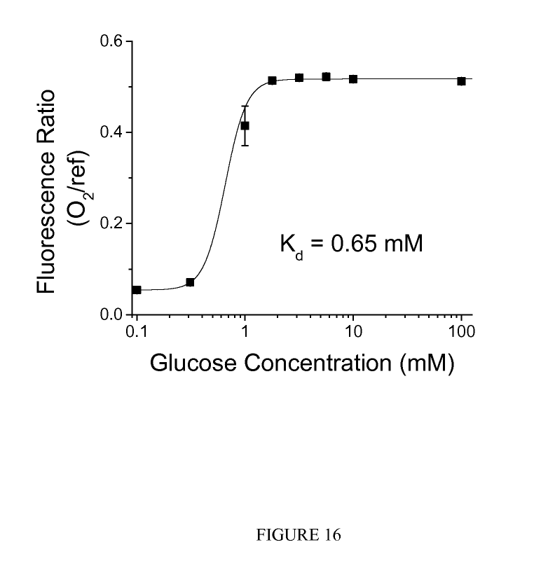

FIG. 16 represents a calibration curve for oxygen nanosensors (with Pt(II) mess-Tetra (pentafluorophenyl)porphine as O₂ sensor dye and octadecyl rhodamine as the reference dye) combined with the catalytic agent glucose oxidase to detect glucose. The polymer used was PVC plasticized with bis-2-ethylhexyl sebacate. Pt and RH 18 ref were dyes used.

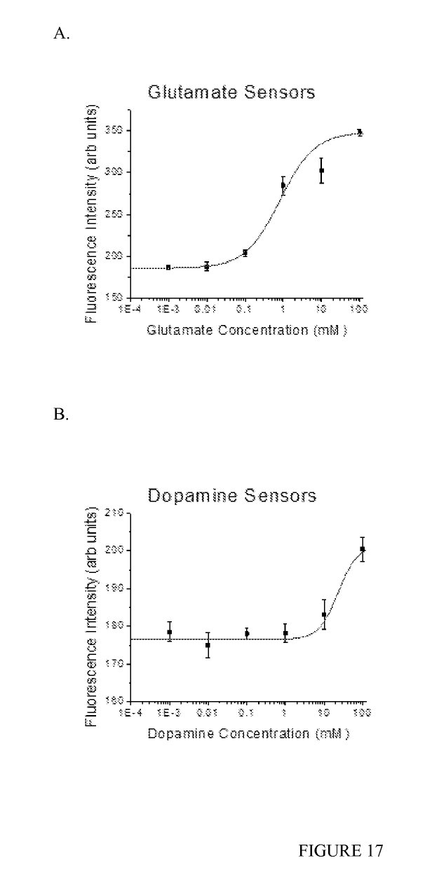

FIGS. 17A-B represent calibration curves similar to FIG. 16 except with no reference dye and different catalytic agents were used; glutamate oxidase was used for glutamate detection, and tyrosinase for dopamine detection.

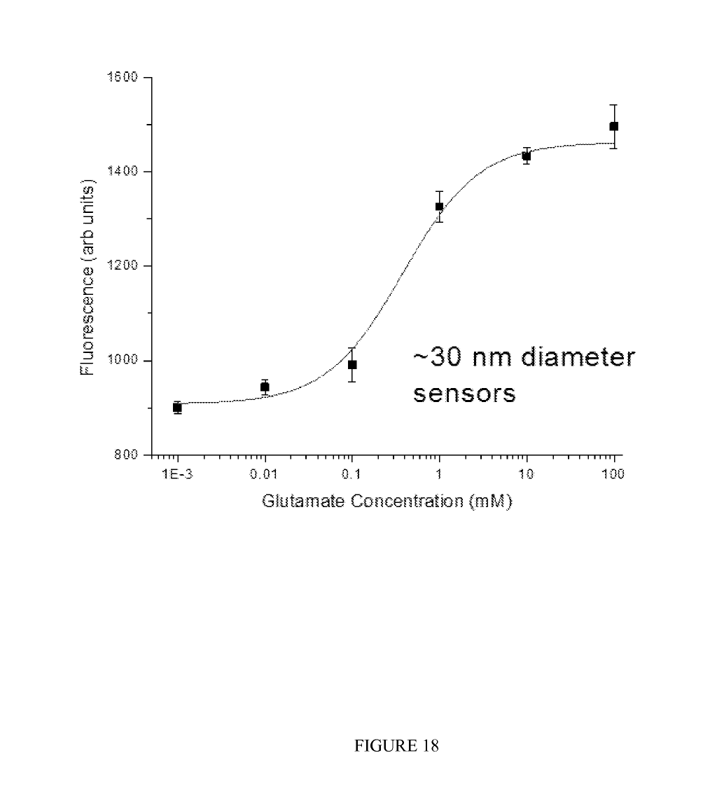

FIG. 18 represents a calibration curve using oxygen-sensitive ultrasmall nanosensors with glutamate oxidase to detect glutamate. The ultrasmall nanosensors are based on plutonic F127 polymer (PEG-block-PPG-block-PEG) and silica with Pt(II) meso-Tetra(pentafluorophenyl)porphine as the dye.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A composition comprising:
a catalytic agent that catalyzes a reaction in which a target substrate and/or a co-substrate is converted into one or more products; and
a nanosensor that is sensitive to an analyte such that the nanosensor emits a fluorescent signal upon detecting the analyte, wherein the analyte is the target substrate, the co-substrate, or at least one of the one or more products.

2. The composition of claim 1, wherein the analyte is selected from the group consisting of oxygen, hydrogen, ammonia, nitrate, nitrite, and sulfate.

3. The composition of claim 2, wherein the nanosensor is sensitive to oxygen.

4. The composition of claim 3, wherein the nanosensor comprises a metal-centered dye, organic dye, or biological molecule.

5. The composition of claim 4, wherein the metal center of the metal-centered dye comprises ruthenium (Ru(phen)3), platinum (Pt(II) meso-Tetra(pentafluorophenyl)porphine), osmium, rhenium, iridium, or mixtures thereof.

6. The composition of claim 1, wherein the nanosensor and the catalytic agent are operably linked.

7. The composition of claim 6, wherein the catalytic agent is diamino oxidase, acetylcholine esterase, glucose oxidase, cholesterol oxidase, or glutamate dehydrogenase.

8. The composition of claim 7, wherein the nanosensor and catalytic agent are embedded in a matrix.

9. The composition of claim 8, wherein the matrix is a hydrogel that allows the target substrate to contact the catalytic agent.

10. The composition of claim 1, wherein the nanosensor and catalytic agent are attached to a surface of a microfluidic device.

11. The composition of claim 10, wherein the nanosensor and catalytic agent are attached to the surface of the microfluidic device through linkers.

12. The composition of claim 1, wherein the nanosensor and the catalytic agent are attached to the surface of a nanodevice.

13. The composition of claim 12, wherein the nanodevice comprises a polymer to which the nanosensor and the catalytic agent are attached.

14. The composition of claim 13, wherein the polymer is polyvinyl chloride, polycaprolactone, polylactic acid, polylactic co-glycolic acid, poly(3-hydroxybutyrate), poly(carboxy phenoxy propane)-(sebacic acid), polypropylene fumarate, poly(alkyl cyanoacrylate, chitosan, alginate, polylysine, collagen, or mixtures thereof.

15. A method of detecting a target substrate, comprising:
(a) contacting a catalytic agent with a target substrate and/or a co-substrate such that the catalytic agent catalyzes conversion of the target substrate and/or the co-substrate into one or more products;
(b) contacting a nanosensor with an analyte such that the nanosensor emits a fluorescent signal upon detecting the analyte, wherein the analyte is the target substrate, the co-substrate, or at least one of the one or more products; and
(c) measuring the concentration of the target substrate based on the fluorescent signal generated by the nanosensor.

16. The method of claim 9, wherein the analyte is selected from the group consisting of oxygen, hydrogen, ammonia, nitrate, nitrite, and sulfate.

17. The method of claim 16, wherein the nanosensor is sensitive to oxygen.

18. The method of claim 17, wherein the nanosensor comprises a metal-centered dye, organic dye, or biological molecule.

19. The method of claim 18, wherein the metal center of the metal-centered dye comprises ruthenium (Ru(phen)3), platinum (Pt(II) meso-Tetra(pentafluorophenyl)porphine), osmium, rhenium, iridium, or mixtures thereof.

20. The method of claim 15, wherein the nanosensor and the catalytic agent are operably linked.

21. The method of claim 20, wherein the catalytic agent is diamino oxidase, acetylcholine esterase, glucose oxidase, cholesterol oxidase, or glutamate dehydrogenase.

22. The method of claim 21, further comprising embedding the nanosensor and catalytic agent in a matrix.

23. The method of claim 22, wherein the matrix is a hydrogel that allows the target substrate to contact the catalytic agent.

24. The method of claim 15, further comprising attaching the nanosensor and catalytic agent to a surface of a microfluidic device.

25. The method of claim 24, wherein the nanosensor and catalytic agent are attached to the surface of the microfluidic device through linkers.

26. The method of claim 15, further comprising attaching the nanosensor and the catalytic agent to a surface of a nanodevice.

27. The method of claim 26, wherein the nanodevice comprises a polymer to which the nanosensor and the catalytic agent are attached.

28. The method of claim 27, wherein the polymer is polyvinyl chloride, polycaprolactone, polylactic acid, polylactic co-glycolic acid, poly(3-hydroxybutyrate), poly(carboxy phenoxy propane)-(sebacic acid), polypropylene fumarate, poly(alkyl cyanoacrylate, chitosan, alginate, polylysine, collagen, or mixtures thereof.
